(19) United States
(12) Patent Application Publication
(10) Pub. No.: US 2020/0324054 A1
(43) Pub. Date: Oct. 15, 2020

(54) Injection Device

(71) Applicant: Sanofi-Aventis Deutschland GMBH, Frankfurt am Main (DE)

(72) Inventors: Michael Helmer, Frankfurt am Main (DE); Michael Schabbach, Frankfurt am Main (DE); Julian Kersting, Rüsselsheim (DE)

(21) Appl. No.: 16/760,782

(22) PCT Filed: Nov. 5, 2018
(86) PCT No.: PCT/EP2018/080100

§ 371 Date: Apr. 30, 2020

(30) Foreign Application Priority Data
Nov. 7, 2017 (EP) 17200315.4

(51) Int. Cl. A61M 5/315

(52) U.S. Cl. CPC A61M 5/31526; A61M 5/3158

(57) ABSTRACT

An injection device for setting and injecting a dose of a medicament comprises an elongated housing extending along a longitudinal axis, a piston rod to operably engage with a piston of a cartridge filled with the medicament, a clutch comprising a hollow interior and configured to be rotated in a first direction relative to the housing during setting of a dose and to be rotated in a second direction relative to the housing during delivery of the dosea driver configured to follow a rotational movement of the clutch in the second direction during delivery of the dose, the piston rod being operably engaged with the driver and configured to be displaced along the longitudinal axis relative to the housing when the driver rotates in the second direction, and a retainer configured to prevent a rotational movement of the driver relative to the housing in the first direction during setting of the dose.

### CROSS REFERENCE TO RELATED APPLICATIONS

The present application is the national stage entry of International Patent Application No. PCT/EP2018/080100, filed on Nov. 5, 2018, and claims priority to Application No. EP 17200315.4, filed on Nov. 7, 2017, the disclosures of which are incorporated herein by reference.

### TECHNICAL FIELD

The present disclosure relates in one aspect to an injection device, such as a pen-type injector for setting and dispensing of a dose of a medicament. In particular, the disclosure relates to an injection device with a longitudinally displaceable slider for setting and dispensing of the medicament. In a further aspect the disclosure relates to a mechanically implemented injection device that provides an automated dose setting in accordance to a preselected size of a dose.

### BACKGROUND

Injection devices for setting and dispensing a single or multiple doses of a liquid medicament are as such well-known in the art. Generally, such devices have substantially a similar purpose as that of an ordinary syringe.

Injection devices, in particular pen-type injectors have to meet a number of user-specific requirements. For instance, with patient's suffering chronic diseases, such as diabetes, the patient may be physically infirm and may also have impaired vision. Suitable injection devices especially intended for home medication therefore need to be robust in construction and should be easy to use. Furthermore, manipulation and general handling of the device and its components should be intelligible and easy understandable. Moreover, the dose setting as well as dose dispensing procedure must be easy to operate and has to be unambiguous.

Typically, such devices comprise a housing including a particular cartridge holder, adapted to receive a cartridge at least partially filled with the medicament to be dispensed. Such devices further comprise a drive mechanism, usually having a displaceable piston rod which is adapted to operably engage with a piston of the cartridge. By means of the drive mechanism and its piston rod, the piston of the cartridge is displaceable in a distal direction or in a dispensing direction and may therefore expel a predefined amount of the medicament via a piercing assembly, which is to be releasably coupled with a distal end section of the housing of the injection device.

The medicament to be dispensed by the injection device is provided and contained in a multi-dose cartridge. Such cartridges typically comprise a vitreous barrel sealed in a distal direction by means of a pierceable seal and being further sealed in proximal direction by the piston. With reusable injection devices an empty cartridge is replaceable by a new one. In contrast, injection devices of disposable type are to be discarded when the medicament in the cartridge has been dispensed or used-up.

Use and operation of the injection device should be simple, failure safe and intuitive. The injection device should provide a precise setting and dispensing of a user-selectable or pre-selected dose of the medicament. The injection device should be robust and should yet enable a cost efficient manufacturing. In one aspect the injection device should be rather compact in terms of its geometric size.

### SUMMARY

In one aspect there is provided an injection device for setting and injecting of a dose of a medicament. The injection device comprises an elongated housing extending along a longitudinal axis. The longitudinal axis may coincide with an axial direction. The injection device further comprises a piston rod to operably engage with a piston or bung of a cartridge. The cartridge is filled with the medicament to be injected by the injection device. The injection device further comprises a clutch. The clutch comprises a hollow interior and is configured to be rotated in a first direction relative to the housing during setting of a dose. The clutch is further configured to be rotated in a second direction relative to the housing during delivery of the dose. The first and the second directions are opposite to each other. Hence, the second direction is opposite to the first direction. The clutch is typically rotatably supported inside the housing.

The injection device further comprises a driver configured to follow a rotational movement or rotation of the clutch in the second direction during delivery of the dose. The piston rod is further operably engaged with the driver and is configured to be displaced along the longitudinal axis relative to the housing when the driver rotates in the second direction. The injection device also comprises a retainer configured to prevent a rotational movement of the driver relative to the housing in the first direction during setting of the dose. Typically, the driver is exclusively rotatable in the second direction. It is generally prevented and hindered to rotate in the first direction. In this way and due to a torque proof engagement between the driver and the piston rod the piston rod is configured to be displaced only and exclusively in one longitudinal direction, typically along the distal longitudinal direction, hence towards the piston or bung of the cartridge.

The driver comprises a driver sleeve section. A portion of the driver sleeve section is arranged inside the hollow interior of the clutch. In this way the driver sleeve section at least partially overlaps with the clutch in longitudinal direction. The driver and the clutch are at least partially arranged in a nested or interleaved configuration thus enabling a reduction of the geometric size of the injection device. The nested and/or interleaved arrangement of at least a portion of the driver and at least a portion of the clutch allows minimizing a geometric size of the arrangement of the driver and the clutch. The installation space for the clutch and the driver can be substantially reduced thus enabling a rather compact design and geometry of the injection device.

The retainer is permanently fixed to the housing or may even belong to the housing. The retainer can be configured as a portion of the housing or may be integrally formed with the housing. In other examples the retainer is a separate component permanently fastened to the housing with regard to both, a rotational and a sliding movement. The retainer may comprise or may form a support for further components of the injection device. In other words, the retainer is positionally fixed to the housing.

The mutual engagement of the clutch and the driver is such that only and exclusively a rotation of the clutch in the second direction leads to a respective rotation of the driver in or in the second direction. An oppositely directed rotation of the clutch, i.e. in the first direction has no influence on the driver because the driver is hindered to rotate in the first direction through the interaction or engagement with the retainer.

The clutch and the driver form a unidirectional torque transmission for delivery of the dose. For a dose setting procedure the clutch is rotatable in the first direction while the driver is hindered to rotate in the first direction. Here, the mutual engagement of the clutch and the driver allows for a rotation of the clutch in the first direction relative to the driver. When the clutch is rotated in the second direction it is in torque-proof engagement with the driver. Hence, a rotation of the clutch in the second direction is directly transferred to the driver in a rather slip-free way.

At least one of the clutch and the driver are longitudinally constrained inside the housing. The driver and/or the clutch may be subject to a rather limited axial or longitudinal displacement relative to the housing, e.g. for temporally disengaging the driver from the retainer. During setting of a dose and/or during dispensing of a dose at least one of the clutch and the driver or both, the clutch and the driver remain substantially axially fixed.

In a further example the driver comprises a first toothed section at an axial face that is configured to engage with a correspondingly-shaped toothed section of the retainer. The first toothed section may face in proximal direction and the correspondingly-shaped toothed section of the retainer may face in distal direction. The toothed section of the retainer may be also provided at an axial end face of the retainer. Likewise, the first toothed section may be provided at an axial end face of the driver. The first toothed section may be provided at a proximal end face and the correspondingly shaped toothed section of the retainer may be provided at a distal end face of the retainer.

Alternatively, the correspondingly-shaped toothed section of the retainer is provided at a portion of the retainer offset from an axial end face of the retainer but faces in an axial direction. Moreover, the first toothed section of the driver does not necessarily have to be provided at an axial end face of the driver. It may be provided on a radially outwardly or radially inwardly extending flange section that is axially offset from an axial end face of the driver. The first toothed section and the correspondingly-shaped toothed section of the retainer may comprise a hirth-like toothing having multiple teeth protruding in axial direction and being separated along a circumference of the driver or of the retainer with grooves between consecutive teeth extending in a radial direction.

The first toothed section and the correspondingly-shaped toothed section of the retainer are configured to provide a torque-proof engagement at least with regard to a rotation of the driver in the first direction relative to the housing. Since the retainer is fixed to the housing the first toothed section and the correspondingly-shaped toothed section of the retainer are configured to prevent or to hinder a rotation of the driver in the first direction.

According to a further example the first toothed section comprises numerous saw teeth protruding in longitudinal direction from the axial face of the driver. The axial face may be an axial end face of the driver. It may be a proximal axial end face or a distal axial end face of the driver. The first toothed section comprises a toothed rim on the axial face comprising numerous saw teeth arranged along the circumference of the annular structure of the toothed section. The saw teeth each comprise a rather steep edge and a shallow edge. The steep edge or flank faces towards the first direction whereas the shallow or rather flat edge of flank faces towards the second direction.

Since the toothed section of the retainer is correspondingly-shaped to the first toothed section of the driver also the correspondingly-shaped toothed section of the retainer comprises numerous saw teeth protruding in longitudinal direction from a respective axial face of the retainer. For instance, the saw teeth of the first toothed section protrude in proximal direction. Accordingly, the saw teeth of the correspondingly-shaped toothed section of the retainer face and extend or protrude in distal direction. The shape and configuration of the saw teeth of the correspondingly-shaped toothed section of the retainer corresponds to the saw toothed profile of the first toothed section. The steep edge of the saw teeth of the toothed section of the retainer face towards the second direction whereas the shallow or flat edge of the saw teeth of the correspondingly-shaped toothed section of the retainer face towards the first direction. In this way the rather steep edges of the saw teeth of the first toothed section and the correspondingly-shaped toothed section of the retainer get in direct abutment thus preventing a rotation of the driver relative to the retainer in the first direction. Along a second direction the shallow or rather flat edges of the saw teeth are allowed to slide relative to each other.

In a further example the injection device comprises a clutch spring that is configured to urge the driver into abutment with the retainer. The clutch spring is particularly configured to longitudinally displace the driver towards the retainer in order to keep the driver and the retainer in torque-proof engagement, at least with regard to a rotation in the first direction. Particularly the clutch spring is configured to urge the driver into abutment with the retainer such that the first toothed section of the driver is kept or brought in abutment with the correspondingly-shaped toothed section of the retainer. By means of the clutch spring the mutually corresponding toothed sections of the clutch and of the retainer are kept in permanent engagement. In this way the driver is effectively hindered to rotate in the first direction relative to the retainer or relative to the housing.

Furthermore and when the first toothed section and the correspondingly-shaped toothed section of the retainer comprise numerous saw teeth, i.e. when the first toothed section and the correspondingly-shaped toothed section of the retainer comprise a saw toothed profile the clutch spring allows and supports a slight axial displacement of the clutch relative to the retainer as the clutch is rotated in the second direction. When rotated in the second direction the shallow or flat edges of the teeth of the first toothed section slide along correspondingly-shaped flat or shallow edges of the correspondingly-shaped toothed section of the retainer.

Due to a non-zero pitch or slope of the shallow edges or flanks of the correspondingly-shaped saw toothed profile the driver is subject to a small but distinct axial displacement relative to the retainer as it is rotated in the second direction. The axial displacement is governed by the axial height of the teeth of the first toothed section and of the correspondingly-shaped toothed section of the retainer. During rotation of the driver relative to the retainer in the second direction the axial displacement of the driver relative to the retainer is at a maximum as the tips of the saw teeth of the first toothed section and the correspondingly-shaped toothed section approach.

As mutually corresponding teeth of the meshing toothed sections pass each other the clutch spring immediately urges the driver into abutment with the retainer such that a tip of the teeth of the first toothed section engage with the correspondingly-shaped grooves in between the saw teeth of the correspondingly-shaped toothed section of the retainer. In effect, a rotation of the driver relative to the retainer in the second direction is accompanied by a slight back and forth sliding displacement of the driver relative to the housing and/or relative to the retainer. As the tips of the mutually corresponding teeth of the first toothed section and the correspondingly-shaped toothed section pass each other an audible click sound is generated thus indicating to the user of the injection device that a dose delivery procedure is in progress. Insofar the interaction between the driver and the retainer provides an audible feedback to a user during delivery of a dose. A rotation of the driver in the first direction relative to the retainer is accompanied by the sliding motion of the shallow edges or flanks of the first toothed section and the correspondingly shaped toothed section thereby inducing an axial displacement of the driver relative to the retainer against the action of the spring.

According to a further example the driver sleeve section comprises a sidewall with a second toothed section on an outside surface of the sidewall. The second toothed section is configured to engage with the clutch so as to transfer a driving torque provided by the clutch towards and onto the driver sleeve section and hence to the driver. Typically, the second toothed section of the driver is arranged and located inside the hollow interior of the clutch. In this way a nested and interleaved engagement of the clutch and the driver is enabled. The second toothed section may be provided adjacent to the first toothed section. The first and the second toothed sections may comprise an identical number of teeth. Moreover, the first and the second toothed sections may comprise a zero phase shift as seen in circumferential direction. In other words, the tips of the teeth of the second toothed section may radially align with the tips of the teeth of the first toothed section.

The second toothed section is configured to mesh and/or to engage with a correspondingly-shaped toothed section or toothed structure provided on an inside surface portion of the hollow interior of the clutch. It may be sufficient when the clutch comprises at least one radially inwardly protruding portion or radially inwardly protruding feature to engage with the second toothed section provided on the outside surface of the driver sleeve section.

According to another example the second toothed section comprises numerous saw teeth protruding radially from the outside surface of the sidewall. The saw teeth protrude radially outwardly from the outside surface of the sidewall. Here, the saw teeth also comprise a steep edge and a shallow or flat edge. Typically, the steep edge faces in the first direction whereas the shallow or flat edge faces in the second direction. The configuration and geometry of the saw teeth of the second toothed section may be substantially identical to the configuration and geometry of the saw teeth of the first toothed section. In particular, the steep edges as well as the shallow or flat edges of the saw teeth of the first toothed section and of the second toothed section may be in radial alignment.

Alternatively, the number of the teeth of the second toothed section may differ from the total number of the teeth of the first toothed section. It is also conceivable, that there is a circumferential phase shift between the teeth of the first toothed section and the teeth of the second toothed section. By means of numerous saw teeth protruding radially from the outside surface of the sidewall a unidirectional engagement between the driver and the clutch can be provided. In this way a rotation of the clutch in the first direction may have no effect on the driver as a rotation of the clutch in the second direction is transferred rather slip free into a corresponding rotation of the driver in the second direction thus leading to a longitudinal displacement of the piston rod in longitudinal direction, typically in longitudinal distal direction for the purpose of expelling a dose of the medicament from the cartridge.

Moreover, the saw teeth or the saw tooth profile of the second toothed section allows a rotation of the clutch in the first direction while the driver is hindered to rotate correspondingly. As the clutch is rotated in the first direction an engaging section of the clutch is and remains in mechanical contact with the second toothed section of the driver. As the engaging section of the clutch slides along or meshes the teeth of the second toothed section an audible sound or a respective click noise is generated thus indicating to a user, that a dose setting procedure is in progress and that the clutch is subject to a stepwise rotation relative to the housing or relative to the driver.

In a further example the clutch comprises at least one engaging section configured or operable for a unidirectional torque proof engagement with a complementary or correspondingly shaped counter-engaging section of the driver. The engaging section of the clutch and the counter-engaging section of the driver are configured to transmit a torque from the clutch towards and onto the driver when the clutch is rotated in the second direction. The unidirectional torque proof engagement is exclusively and only configured to transfer a torque from the clutch to the driver when the clutch is rotated in the second direction. When the clutch is rotated in the first direction, hence opposite to the second direction the unidirectional torque proof engagement is inoperable to transfer a respective torque or angular momentum.

The engaging section of the clutch faces towards the hollow interior of the clutch. The engaging section may protrude from a sidewall of the clutch and may extend radially inwardly into the hollow interior. It may extend towards the driver sleeve section located inside the hollow interior of the clutch. Correspondingly, the counter-engaging section of the driver may be located on an outside section of the driver sleeve section. The counter-engaging section is hence located at a radial outside surface of the driver sleeve section. It may protrude from a sleeve section of the driver.

The engaging section of the clutch and the counter engaging section of the driver radially overlap, wherein the radial direction refers to an imaginary axis of rotation extending parallel to the longitudinal axis of the elongated housing.

According to another example the clutch comprises at least one engaging section that is configured for a unidirectional torque-proof engagement with the second tooth profile when the clutch is rotated in the second direction. The at least one engaging section of the clutch comprises at least one engaging feature, typically extending or protruding radially inwardly from the clutch so as to engage in a torque-proof manner with the second toothed section of the driver. Typically, the at least one engaging section is configured to abut and to engage with the steep edge of the saw teeth of the second toothed section.

In this way and since the steep edges of the saw teeth of the second toothed section face towards the first direction a torque proof or torque transmitting engagement between the at least one engaging section and the saw teeth of the second toothed section with regard to a rotation in the second direction can be provided. A rotation of the at least one engaging section towards and into the second direction leads to an abutment of the engaging section with the steep edges of the saw teeth of the second toothed section. Since the steep edges may extend in radial direction the engaging sections will remain in abutment as they are rotated in the second direction. In this way a unidirectional torque transmitting engagement between the clutch and the driver is provided.

According to a further example the clutch comprises at least one ratchet member resiliently deformable in a radial direction. The at least one engaging section is arranged at a free end of the at least one ratchet member. The ratchet member may be pre-tensed or biased radially inwardly. The ratchet member may be elastically or resiliently deformable radially outwardly against a restoring force. In this way the ratchet member remains in torque-proof engagement with the steep edges of the saw teeth of the second toothed section when rotated in the second direction. When rotated in the first direction the at least one ratchet member is allowed to slide relative to the second toothed section. Here, the at least one engaging section and the at least one ratchet member are allowed to slide along the shallow or flat edges of the saw toothed profile of the second toothed section. This may generate an audible sound or repetitive click noises thus indicating to the user that a dose setting procedure is in progress.

By providing the at least one engaging section at a free end of the resiliently deformable ratchet member a rather large radial displacement of the engaging section can be provided already with a comparatively small degree of radial deformation of the ratchet member relative to the clutch. By arranging the at least one engaging section at or near a free end of the ratchet member a kind of a leverage effect is provided.

In general, and as an alternative the ratchet member does not need to be resiliently deformable. Instated as an alternative it may be pivotally supported, e.g. on the clutch or on a clutch sleeve sleeve section of the clutch. It may be pivotally supported against a restoring force of a spring.

In another example the clutch comprises a clutch sleeve section enclosing at least a portion of the driver sleeve section of the driver. By means of a clutch sleeve section the clutch comprises at least a portion with a tubular or cylindrical shape. A closed tubular or cylindrical shape provides a substantial stiffness or rigidity to the clutch and to the clutch sleeve section. In this way the clutch is rather robust and is thus capable to provide a sufficient transmission or transfer of angular momentum towards the driver. The same is also valid for the driver and the driver sleeve section. Moreover, the at least partial axial overlapping arrangement of the driver and the clutch and/or an at least partial axial overlapping arrangement of the driver sleeve section with the clutch sleeve section provide a rather stable and robust arrangement and mechanical interaction between the clutch and the driver.

In a further example the at least one ratchet member comprises an arc-shaped geometry that is conformal to a sidewall of the clutch sleeve section. The at least one ratchet member may be provided at an axial end of the clutch sleeve section. Alternatively, the clutch sleeve section may comprise a U-shaped slit extending along the outer circumference of the clutch sleeve section thus providing an arc-shaped and resiliently deformable section of the clutch thus forming the resiliently deformable ratchet member.

The arc-shaped geometry of the ratchet member comprises a curvature that is substantially identical to the curvature of the clutch sleeve section. In this way the total radial dimensions of the clutch and its ratchet members can be kept at a minimum.

In another example the clutch comprises at least a first ratchet member and a second ratchet member. The first ratchet member and the second ratchet member are arranged geometrically opposite to each other. Furthermore it is conceivable, that the clutch comprises numerous, e.g. at least three or even four ratchet members that are equidistantly spaced with regard to the outer circumference of the clutch or of the clutch sleeve section. In this way and since the second toothed section on the outside surface of the sidewall of the driver comprises a closed ring structure or an annular structure not only one but at least two ratchet members may be in torque-proof engagement with the second toothed section of the driver at a time.

It is conceivable, that the circumferential distance between the first and the second ratchet members matches with the periodicity of the second toothed section on the outside surface of the driver. As the clutch is rotated in the first direction relative to the driver the first and the second ratchet members simultaneously engage and disengage with consecutive teeth of the second toothed section. When the clutch is rotated in the second direction and hence during delivery of the dose the first and the second ratchet members are both in a torque-proof engagement with a steep edge of the saw tooth profile of the second toothed section. In this way the angular momentum provided by the clutch can be distributed to the first and second ratchet members, each of which transferring a respective portion of the total angular momentum to the driver.

The angular momentum to be transferred from the clutch to the driver may be distributed between the at least first and the at least second ratchet members. The mechanical load on the first and on the second ratchet member can be substantially reduced in this way thus enabling a further miniaturization of the first and second ratchet members. Moreover, the failure safety of the first and second ratchet members can be improved in this way. If there should be provided even more ratchet members, e.g. three or four ratchet members they are distributed equidistantly along the circumference of the clutch so as to homogeneously distribute the angular momentum to be transferred from the clutch to the driver.

In a further example one of the driver and the housing is threadedly engaged with the piston rod and the other one of the driver and the housing is in splined engagement with the piston rod. A splined engagement is configured and operable to transfer angular momentum but provides an axial sliding displacement between the respective components that are in splined engagement. The splined engagement comprises a rotational lock between the components that are splined engaged. Typically, the splined engagement comprises one protrusion on one of the components in engagement with a longitudinal groove provided on the other of the components.

As an example, the driver is threadedly engaged with the piston rod and the piston rod is in splined engagement with the housing. Here, the driver comprises an inner thread coupled or engaged with an outer thread of the piston rod. The piston rod further comprises an elongated groove that is engaged with a radially inwardly extending pin or protrusion of the housing. In this way, the piston rod is rotationally locked to the housing but is slidably displaceable relative to the housing as the driver, which is typically rotationally supported relative to the housing but axially fixed to the housing, is rotated.

In a further example the driver is rotationally locked to the piston rod. The piston rod in turn is threadedly engaged with the housing. In the present context rotationally locked means that the driver is hindered to rotate relative to the piston rod. Hence, any rotation of the driver is unalterably transferred to the piston rod. For instance, the piston rod comprises a longitudinally extending groove and the driver comprises a protrusion that is engaged with the longitudinal groove of the piston rod. Typically, the driver comprises a bore through which the piston rod extends in longitudinal direction. The piston rod extends through the driver sleeve section and the driver sleeve section comprises at least one radially inwardly extending protrusion in permanent torque-proof engagement with the longitudinal groove on the outside surface of the piston rod.

The longitudinal groove of the piston rod may intersect an outer threaded portion of the piston rod that is in permanent threaded engagement with the housing. In this way the piston rod is allowed to slide in longitudinal direction relative to the driver. The piston rod may advance in distal longitudinal direction as it is subject to a rotational movement relative to the housing. While the piston rod is rotated through the interaction with the driver in the second direction it advances in distal direction due to the threaded engagement with the housing. At the same time the piston rod is subject to a longitudinal sliding displacement relative to the driver while the driver rotates in unison with the piston rod and remains axially constrained inside the housing. In short, the driver and the piston rod are in a so called splined engagement by way of which a rotation of the driver is transferred into a rotation of the piston rod but which engagement allows for a relative sliding longitudinal displacement between the driver and the piston rod.

In another example the clutch comprises a threaded section that is in threaded engagement with a slider. The slider is longitudinally slidably displaceable relative to the housing. The slider is secured against rotational movement relative to the housing. In other words, the slider is rotationally locked to the housing. The threaded engagement between the slider and the clutch is such, that a longitudinal sliding displacement of the slider transfers into a respective rotation of the clutch relative to the housing while the clutch remains axially constrained in the housing.

The threaded section of the clutch is typically provided on an outside surface of the clutch, typically on an outside surface of the clutch sleeve section. The threaded engagement between the clutch and the slider is configured such that a distally directed sliding displacement of the slider relative to the housing transfers into a rotation of the clutch in the second direction. As the slider is subject to a proximally directed displacement relative to the housing the clutch is rotated in the opposite direction, i.e. in the first direction. Typically, the slider comprises a dose button or is in axial engagement with a dose button that may protrude from a proximal end of the housing.

Typically, the slider is displaceable relative to the housing from an initial position in a proximal direction towards at least a first activation position for setting of the dose. Such a proximally directed sliding displacement is accompanied by a rotation of the clutch in the first direction. The slider is longitudinally displaceable relative to the housing in the distal direction from the at least first activation position towards the initial position for dispensing of the dose. In the at least first activation position the slider, a portion thereof or a further component in axial engagement with the slider protrudes proximally from a proximal end of the housing. In this configuration the slider or a dose button in axial engagement with the slider is depressible by a user, e.g. by a thumb of a user in distal direction thus urging the slider back into the initial position.

This sliding motion of the slider relative to the housing transfers into a respective rotation of the clutch in the second direction. The rotation of the clutch is transferred into a respective rotation of the driver and hence into a distally directed advancing sliding motion of the piston rod thereby displacing the bung of the cartridge further in distal direction so as to expel a predefined amount of the medicament from the cartridge.

The slider may comprise a sleeve-like shape or may comprise at least two elongated legs or arms that are provided with a threaded section at an inside-facing sidewall to engage with the threaded section on the outside of the clutch.

In a further example, the slider is axially biased relative to the housing by a spring. Typically, the slider is biased in longitudinal proximal direction by the spring. The slider is slidably displaceable relative to the housing under the action of the spring. The slider is displaceable relative to the housing in the longitudinal distal direction against the action of the spring.

Moreover, the slider is slidably displaceable in longitudinal direction relative to the housing from a retracted or initial position towards an activation position. The activation position of the slider is proximally shifted compared to the initial or retracted position. When in the activated position the slider itself or a dose button operably connected to the slider may protrude from the proximal end of the housing.

The slider may be engageable or may be engaged with an interlock that is configured to keep the slider in a retracted, hence distal position relative to the housing. Upon release of the interlock, the slider is displaceable in proximal direction relative to the housing under the action of the spring. The spring-induced sliding displacement of the slider relative to the housing is effective to induce a rotation onto the clutch along the first direction. In this way, the spring and the slider provide an automated dose setting of the injection device. A dose setting procedure can be initiated simply by releasing the slider and by allowing and supporting a proximally directed sliding motion of the slider relative to the housing.

In a further example the driver comprises a flange protruding radially outwardly from the driver sleeve section. The flange is in axial abutment with an axial face, typically with an axial end face of the clutch. In this way the clutch can be axially supported on the flange of the driver.

The flange may provide a twofold function. The radially widened geometry of the flange may provide and act as a support face for the clutch spring. One end of the clutch spring may be in abutment with the flange of the driver. Another end of the clutch spring may be supported on a respective axial face or protrusion of the housing. An opposite side of the flange may be in axial abutment with the clutch. In this way the clutch and the driver are axially biased by the clutch spring. For instance, the clutch spring is in axial abutment with a distal-facing side of the flange of the driver thus urging the driver in proximal direction into abutment with the retainer.

The clutch may be axially sandwiched between the flange of the driver and the retainer. When the slider is subject to a displacement towards the distal direction, e.g. during dispensing of a dose the clutch as well as the flange and the driver in abutment with the clutch may be subject to an initial but slight distally directed sliding displacement by way of which the clutch spring is axially squeezed and by way of which the driver is displaced in distal direction so that it is disengaged from the retainer. In this way the driver may be allowed to rotate in the second direction without any interaction with the retainer. Expelling or delivery of the dose may then be void of an audible click noise but a dispensing force required to displace the piston rod in distal direction may be reduced since there is now a reduced or no frictional engagement between the saw teeth of the first toothed section of the driver and the correspondingly-shaped toothed section of the retainer during a dose dispensing procedure.

In another example the injection device further comprises a cartridge. The cartridge comprises a barrel filled with the medicament. The barrel is sealed by a bung or piston that is axially displaceable relative to the barrel by means of the piston rod. For and during a dispensing operation the piston rod is operably engageable with the bung of the cartridge in order to displace the bung in a distal direction. Typically, a distal end of the cartridge is sealed by a pierceable membrane, such as a septum. For dispensing of the medicament the pierceable seal can be penetrated by a double-tipped injection needle. A distally directed displacement of the bung induced by a correspondingly advancing piston rod therefore leads to the expelling of the dose of the medicament.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The term “drug” or “medicament”, as used herein, means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, an enzyme, an antibody or a fragment thereof, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exendin-3 or exendin-4 or an analogue or derivative of exendin-3 or exendin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

H-(Lys)4-des Pro36, des Pro37 Exendin-4(1-39)-NH2,

H-(Lys)5-des Pro36, des Pro37 Exendin-4(1-39)-NH2,

des Pro36 Exendin-4(1-39),

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39); or

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39),

wherein the group -Lys6-NH2 may be bound to the C-terminus of the Exendin-4 derivative;

or an Exendin-4 derivative of the sequence

des Pro36 Exendin-4(1-39)-Lys6-NH2 (AVE0010),

H-(Lys)6-des Pro36 [Asp28] Exendin-4(1-39)-Lys6-NH2,

des Asp28 Pro36, Pro37, Pro38Exendin-4(1-39)-NH2,

H-(Lys)6-des Pro36, Pro38 [Asp28] Exendin-4(1-39)-NH2,

H-Asn-(Glu)5des Pro36, Pro37, Pro38 [Asp28] Exendin-4(1-39)-NH2,

des Pro36, Pro37, Pro38 [Asp28] Exendin-4(1-39)-(Lys)6-NH2,

H-(Lys)6-des Pro36, Pro37, Pro38 [Asp28] Exendin-4(1-39)-(Lys)6-NH2,

H-Asn-(Glu)5-des Pro36, Pro37, Pro38 [Asp28] Exendin-4(1-39)-(Lys)6-NH2,

H-(Lys)6-des Pro36 [Trp(O2)25, Asp28] Exendin-4(1-39)-Lys6-NH2,

H-des Asp28 Pro36, Pro37, Pro38 [Trp(O2)25] Exendin-4(1-39)-NH2,

H-(Lys)6-des Pro36, Pro37, Pro38 [Trp(O2)25, Asp28] Exendin-4(1-39)-NH2,

H-Asn-(Glu)5-des Pro36, Pro37, Pro38 [Trp(O2)25, Asp28] Exendin-4(1-39)-NH2,

des Pro36, Pro37, Pro38 [Trp(O2)25, Asp28] Exendin-4(1-39)-(Lys)6-NH2,

H-(Lys)6-des Pro36, Pro37, Pro38 [Trp(O2)25, Asp28] Exendin-4(1-39)-(Lys)6-NH2,

H-Asn-(Glu)5-des Pro36, Pro37, Pro38 [Trp(O2)25, Asp28] Exendin-4(1-39)-(Lys)6-NH2,

H-(Lys)6-des Pro36 [Met(O)14, Asp28] Exendin-4(1-39)-Lys6-NH2,

des Met(O)14 Asp28 Pro36, Pro37, Pro38 Exendin-4(1-39)-NH2,

H-(Lys)6-desPro36, Pro37, Pro38 [Met(O)14, Asp28] Exendin-4(1-39)-NH2,

H-Asn-(Glu)5-des Pro36, Pro37, Pro38 [Met(O)14, Asp28] Exendin-4(1-39)-NH2,

des Pro36, Pro37, Pro38 [Met(O)14, Asp28] Exendin-4(1-39)-(Lys)6-NH2,

H-(Lys)6-des Pro36, Pro37, Pro38 [Met(O)14, Asp28] Exendin-4(1-39)-(Lys)6-NH2,

H-Asn-(Glu)5 des Pro36, Pro37, Pro38 [Met(O)14, Asp28] Exendin-4(1-39)-(Lys)6-NH2,

H-Lys6-des Pro36 [Met(O)14, Trp(O2)25, Asp28] Exendin-4(1-39)-Lys6-NH2,

H-des Asp28 Pro36, Pro37, Pro38 [Met(O)14, Trp(O2)25] Exendin-4(1-39)-NH2,

H-(Lys)6-des Pro36, Pro37, Pro38 [Met(O)14, Asp28] Exendin-4(1-39)-NH2,

H-Asn-(Glu)5-des Pro36, Pro37, Pro38 [Met(O)14, Trp(O2)25, Asp28] Exendin-4(1-39)-NH2,

des Pro36, Pro37, Pro38 [Met(O)14, Trp(O2)25, Asp28] Exendin-4(1-39)-(Lys)6-NH2,

H-(Lys)6-des Pro36, Pro37, Pro38 [Met(O)14, Trp(O2)25, Asp28] Exendin-4(S1-39)-(Lys)6-NH2,

H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exendin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (˜150 kDa) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, δ, ϑ, γ, and μ. The type of heavy chain present defines the isotype of antibody; these chains are found in IgA, IgD, IgE, IgG, and IgM antibodies, respectively.

Distinct heavy chains differ in size and composition; α and γ contain approximately 450 amino acids and δ approximately 500 amino acids, while μ and ε have approximately 550 amino acids. Each heavy chain has two regions, the constant region (C_{H}) and the variable region (V_{H}). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of all antibodies is very similar, the unique property of a given antibody is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H—H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

It will be further apparent to those skilled in the art that various modifications and variations can be made to the present injection device without departing from the spirit and scope of the what is defined in the appended claims. Further, it is to be noted, that any reference numerals used in the appended claims are not to be construed as limiting the scope of the disclosure.

### BRIEF DESCRIPTION OF THE FIGURES

In the following, embodiments of the injection device are described in detail by making reference to the drawings, in which:

FIG. 1 shows a schematic illustration of a pen-type injection device,

FIG. 2 is an exploded view of components of the injection device of FIG. 1,

FIG. 3 shows an isolated perspective view of a drive mechanism of the injection device,

FIG. 4 is another perspective view of the drive mechanic with a preselector,

FIG. 5 shows the drive mechanism according to FIG. 4 with the slider in an activation position,

FIG. 6 is an isolated perspective view of the piston rod, a threaded insert and a driver,

FIG. 7 is another perspective view of FIG. 6 as seen from the proximal end,

FIG. 8 is an isolated perspective view of a clutch,

FIG. 9 is a perspective view of the components of FIG. 6 together with the clutch,

FIG. 10 is a side view of the arrangement according to FIG. 9 when arranged inside the housing of the injection device but without the slider,

FIG. 11 is another side view of the arrangement according to FIG. 10 inclusing the slider,

FIG. 12 is a side view of the drive mechanism,

FIG. 13 is an isolated perspective view of a support fixed inside the housing of the injection device,

FIG. 14 is an isolated perspective view of the slider,

FIG. 15 shows the preselector,

FIG. 16 is a perspective view of the spring,

FIG. 17 is an enlarged side view of the interlock,

FIG. 18 shows the proximal end of the injection device with the slider in the initial position,

FIG. 19 shows the proximal end of the injection device with the slider in an activation position,

FIG. 20 shows the proximal end of the injection device with the slider in another activation position,

FIG. 21 shows the proximal end of the injection device with the slider in a further activation position,

FIG. 22 is a longitudinal cross-section through the injection device and

FIG. 23 is a longitudinal cross-section of the injection device when rotated by 90° compared to the cross-section of FIG. 22.

### DETAILED DESCRIPTION

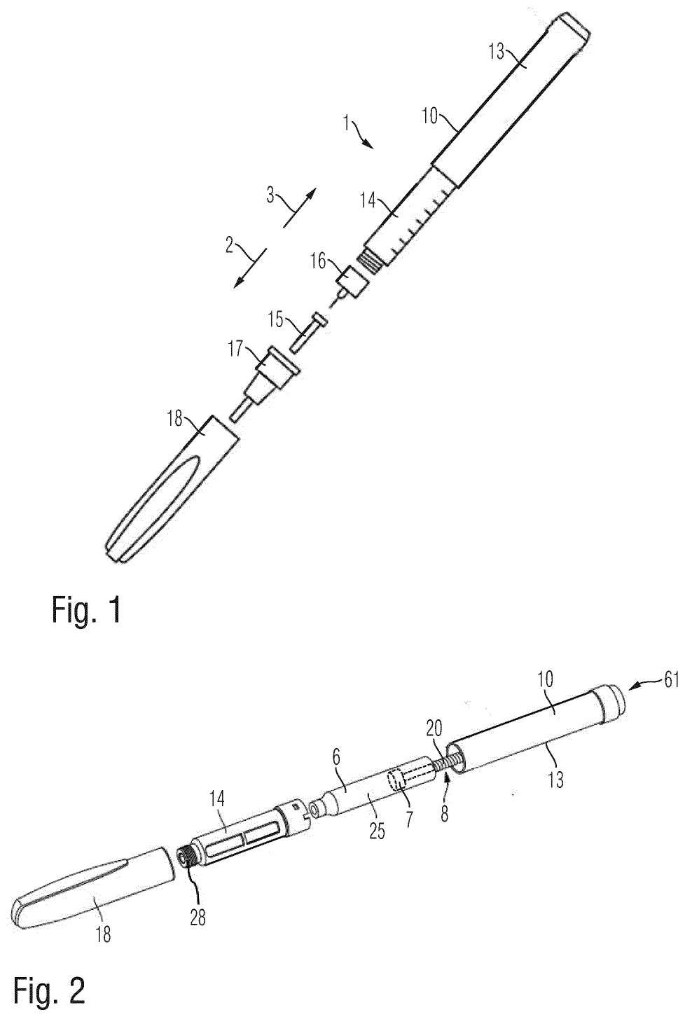

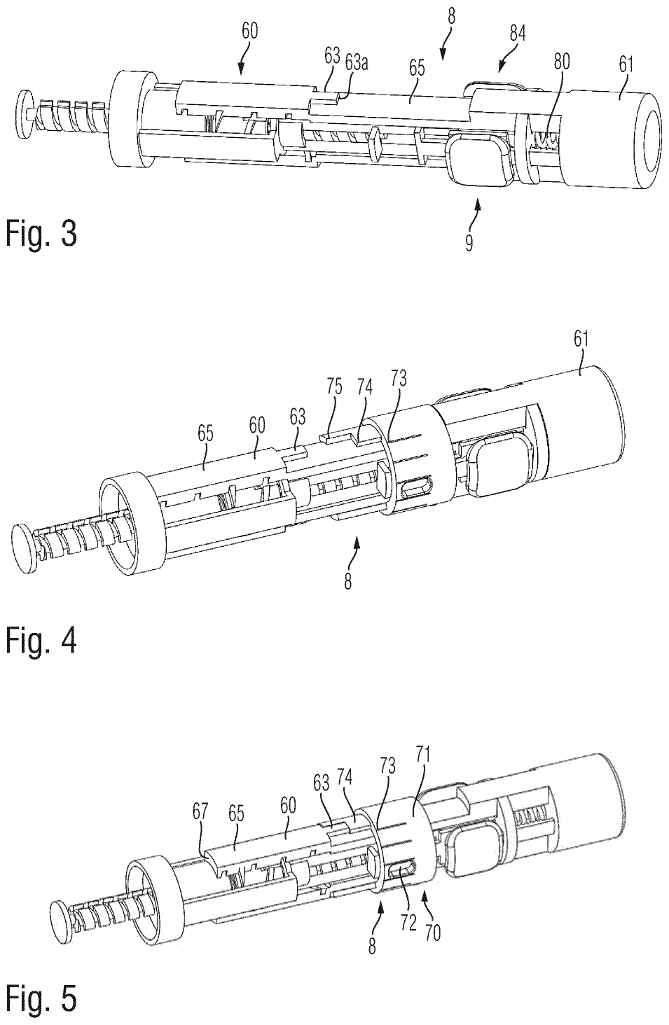

The injection device 1 as shown in FIG. 1 comprises a housing 10 of tubular and elongated shape. The injection device 1 may be configured as a prefilled disposable injection device. Alternatively, it may be configured as a reusable injection device.

The injection device 1 comprises a distal end to which a needle assembly 15 can be affixed. An injection needle of the needle assembly 15 can be protected by an inner needle cap 16 and further by an outer needle cap 17. The distal end of the injection device 1 is further covered by a protective cap 18 that is releasably engageable with the housing 10 of the injection device 1. When attached to the injection device 1 the protective cap 18 covers a portion of the housing of the injection device 1 that is also denoted as a cartridge holder 14. The cartridge holder 14 is configured to accommodate a cartridge 6 filled with a medicament. The cartridge 6 comprises a tubular-shaped barrel 25. The barrel 25 is sealed in distal direction 2 by means of a pierceable seal 26.

Towards the proximal direction 3 the barrel 6 is sealed by a displaceable piston 7. The piston 7 is displaceable in distal direction 2 by means of a piston rod 20 of a drive mechanism 8 of the injection device 1 expelling a predefined amount of the medicament from the cartridge and through the injection needle of the needle assembly 15. The pierceable seal 26 is configured as a septum and is pierceable by a proximally directed tipped end of the needle assembly 15. Furthermore, the cartridge holder 14 comprises a threaded socket 28 at its distal end to threadedly engage with a correspondingly threaded portion of the needle assembly 15. By attaching the needle assembly 15 to the distal end of the cartridge holder 14 the seal 26 of the cartridge 6 is penetrated thereby establishing a fluid transferring access to the interior of the cartridge 6.

The proximal portion or the main housing 10 of the injection device 1 is configured to house and to accommodate a drive mechanism 8 the entirety of which is illustrated in FIGS. 4 and 5. Here, the drive mechanism 8 is a combined drive mechanism 8 and dose setting mechanism 9. The drive mechanism 8 is configured to set and to dispense a dose of the medicament. Here, the drive mechanism 8 may coincide with a dose setting mechanism 9. In the following reference is made to the drive mechanism 8.

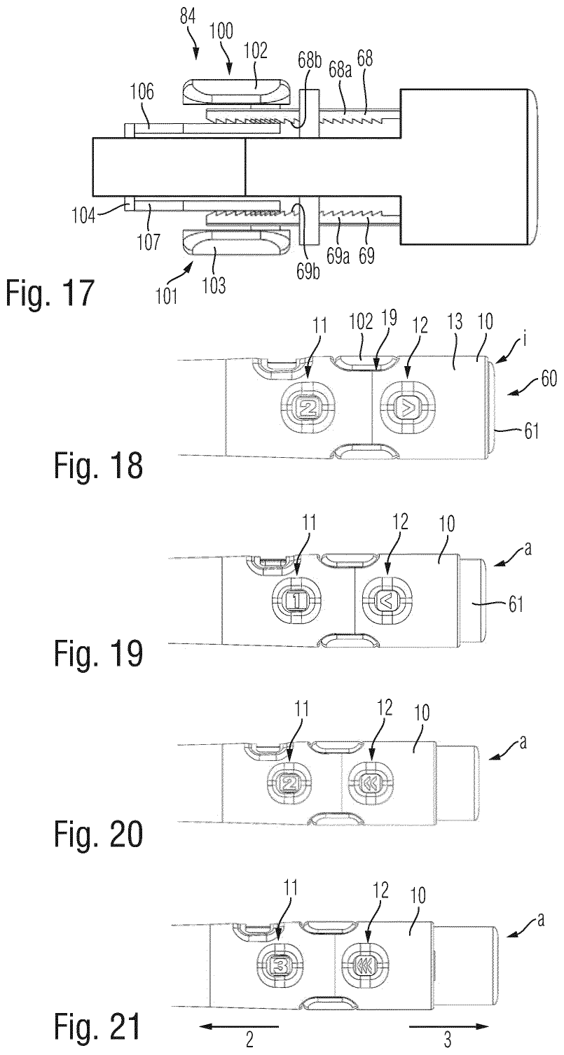

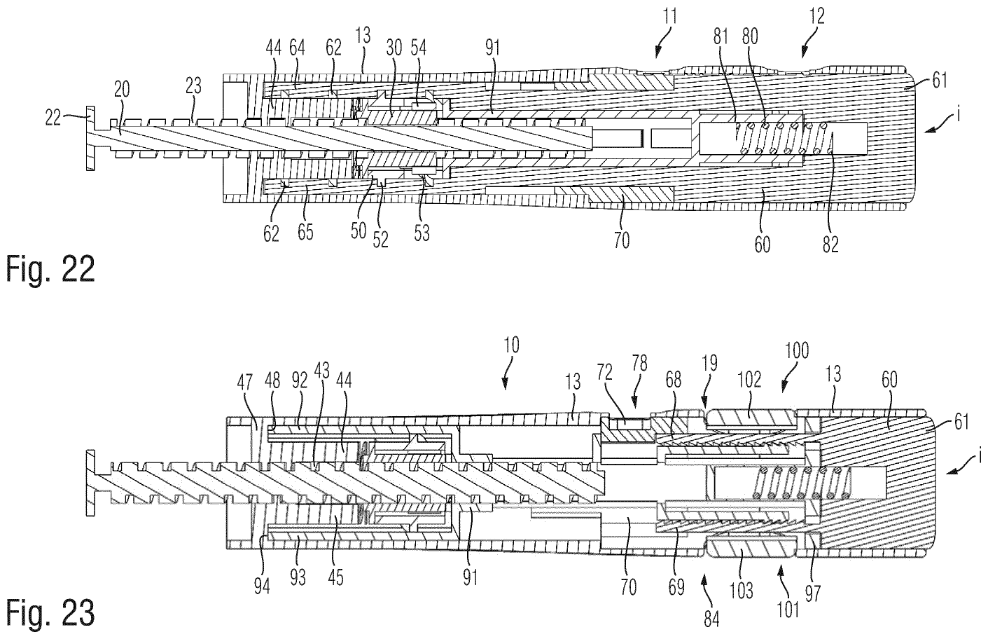

Operation of the injection device 1 is rather simple. For setting of a dose a user has to trigger a release member 100, 101, in form of a first and a second release button 102, 103. As illustrated in FIG. 17 the release members 100, 101 each comprise a release button 102, 103 that is located in a recess 19, hence in a through opening of a sidewall 13 of the housing 10 as indicated in FIG. 23. The release member 100, 101 belong to an interlock 84 that is configured to keep a slider 60 in a retracted position or in an initial position i as shown in FIG. 18. The slider 60 comprises a dose button 61 that is substantially flush with a proximal end face of the housing 10 when in the initial position i. The slider 60 is biased in proximal direction 3 by a spring 80 as shown in FIG. 16. By activating the at least one release member, typically, e.g. by depressing both release members 100, 101 simultaneously the interlock 84 between the slider 60 and the housing 10 is deactivated or abrogated and the slider 60 is free to become displaced in proximal direction 3 under the action of the spring 80. The slider 60 is in slidably engagement with the housing 10. It is prevented from rotating relative to the housing 10. The slider 60 is configured to slide from the initial position i towards the activation position a as it is apparent from a comparison of FIG. 18 and FIG. 19.

In the activation position a as shown in FIG. 19 the slider 60 is depressible, e.g. by a thumb of a user in distal direction 2 so as to advance the piston rod 20 in the distal direction 2 for displacing the piston 7 relative to the cartridge 6. In this way a predefined amount of the medicament can be expelled from the cartridge 6. For dispensing of the dose the slider 60 is operably engaged with a piston rod 20. The drive mechanism 8 serves to transfer a distally advancing sliding motion of the slider 60 into a rotational movement of the piston rod 20, which due to a threaded engagement with the housing 10 advances in distal direction 2 accordingly.

When the slider 60 or the dose button 61 returns into the initial position as illustrated in FIG. 18 the interlock 84 is automatically reactivated so as to keep the slider 60 in the initial position i against the action of the spring 80. A distally directed displacement of the slider 60 acts against the force exerted by the spring 80. The spring 80 is hence biased or tensed as the slider 60 is displaced in distal direction 2. When returning and arriving at the initial position as illustrated in FIG. 18 the interlock 84 engages or re-engages. A repeated depression of at least one, typically of both release members 100, 101 disengages the interlock 84 and enables a repeated displacement of the slider 60 relative to the housing 10 in proximal direction 3 towards the activation position a.

The length of a displacement path of the slider 60 relative to the housing 10 between the initial position i as shown in FIG. 18 and one of the activation positions as shown in FIGS. 19 to 21 is correlated to the size of the dose actually set. The more the slider 60 and the dose button 61 protrude from the proximal end of the housing 10 the larger is the size of the dose to be dispensed in the subsequent dose dispensing procedure.

In order to vary a size of a dose the injection device 1 and hence the drive mechanism 8 comprises a preselector 70 as illustrated in FIGS. 12 and 15. The preselector 70 is at least one of longitudinally or rotationally displaceable relative to the housing 10. It is translationally or rotationally displaceable relative to the housing 10 between at least two preselection positional states. With the example as currently illustrated the preselector 70 is rotatable relative to the housing 10. It is axially fixed to the housing 10. In any of at least two preselection positional states the preselector 70 is fixable to the housing 10. For this, the mutual engagement of the preselector 70 and the housing 10 may comprise a ratchet mechanism, such as at least one protrusion mechanically engageable with one of at least two or more recesses of corresponding shape.

The preselector 70 comprises a sleeve section 71. It is arranged inside the housing 10. An outside facing portion of the sleeve section 71 faces an inside facing portion of the sidewall 13 of the housing 10. The housing 10 comprises a preselection window 11 as illustrated in FIGS. 18 to FIG. 21. On the outside surface of the sleeve section 71 of the preselector 70 there is provided at least one preselection indication 77, e.g. in form of one or several dose indicating numbers, such as 1, 2, 3. Depending on the rotational state of the preselector 70 relative to the housing 10 only one of the dose indicating numbers shows up in the preselection window 11. As shown in FIG. 19 a dose of size 1 is currently preselected. In FIG. 20 a dose of size 2 is preselected and in the configuration of FIG. 21 a dose size characterized by a number 3 is preselected.

The numbers or any other type of preselection indication, such as symbols or letters may represent several standard units of the medicament to be dispensed. For instance, a number 1 of a preselection indication 77 may represent 10 standard units of the medicament. For moving and for rotating the preselector 70 there is provided a radial recess 72 in the outside facing surface of the sleeve section 71. The recess 72 is aligned with a through opening 78 in the sidewall 13 of the housing 10 as illustrated in FIG. 23. Here, authorized persons, such as caregivers may use a tool to reach through the through opening 78 and to engage with the recess 72 of the sleeve section 71. Then, and by making use of the tool the preselector 70 can be rotated with regards to the longitudinal axis z of the elongated housing 10 as an axis of rotation. Consequently, another preselection indication 77 will show up in the preselection window 11. The through opening 78 as shown in FIG. 23 may be covered by a label, an adhesive tape or by a detachable cover so as to prevent unauthorized manipulation of the preselector 70.

As illustrated further in FIG. 15 the preselector 70 comprises numerous preselector stop features 73, 74, 75. The preselector stop features 73, 74, 75 extend in longitudinal direction and may protrude from the sleeve section 71 in distal direction 2. The preselector stop features 73, 74, 75 may be provided as stepped sections of a protrusion 76 that protrude axially or longitudinally from the sleeve section 71 of the preselector 70.

The stop features 73, 74, 75 to be denoted as a first stop feature 73, as a second stop feature 74 and as a third stop feature 75 each comprise a respective stop face 73a, 74a, 75a. The stop faces 73a, 74a, 75a face in distal direction 2. The stop features 73, 74, 75 are configured to engage with a correspondingly shaped dose stop feature 63 of the slider 60. The dose stop feature 63 comprises a proximally facing stop face 63a.

In an initial configuration as illustrated in FIG. 4 there is a longitudinal distance and a free space between the dose stop feature 63 and any of the preselector stop feature 73, 74, 75. This configuration represents the initial position of the slider 60. As the interlock 84 is released the slider 60 is subject to a proximally directed advancing motion under the action of the relaxing spring 80. The slider 60 is subject to the longitudinal movement until the stop face 63a of the dose stop feature 63 gets in axial abutment with one stop face 73a, 74a, 75a of one of the preselector stop features 73, 74, 75.

In the configuration as shown in FIG. 5 the dose stop feature 63 is in axial engagement and axial abutment with the second preselector stop feature 74. The proximally facing stop face 63a is in direct abutment with the distally facing stop face 74a. Since the stop features 63, 73, 74, 75 are located in a common radial plane and since the slider 60 is in sliding engagement with the housing 10 the maximum size of the dose and hence the activation position of the slider 60 is governed by the longitudinal alignment of the dose stop feature 63 with one of the preselector stop features 73, 74, 75. Each stop feature 73, 74, 75 comprises a stop face 73a, 74a, 75a, wherein the stop faces of the various stop features 73, 74, 75 are axially offset with respect to each other.

As illustrated in FIG. 15 the various preselector stop features 73, 74, 75 comprise different elongations in longitudinal or axial direction. Accordingly or alternatively, the stop faces 73a, 74a, 75a of the stop features 73, 74, 75 are located at an axial offset with respect to each other. If for instance the preselector 70 is rotated relative to the housing 10 in such a way that the distalmost stop feature 75 is aligned with the dose stop feature 63 the displacement path of the slider 60 is comparatively short as seen in proximal direction 3 until the dose stop feature 63 gets in axial abutment with the respective stop feature 75.

If another preselector stop feature, such as the preselector stop feature 73 is in longitudinal alignment with the dose stop feature 63 the movement of the slider 60 from the initial position towards the activation position as illustrated in FIG. 21 is rather long, which corresponds to a maximum dose size. When the distal most preselector stop feature 75 is longitudinally aligned with the dose stop feature 63 the smallest preselection indication 77, i.e. number 1 shows up in the preselection window 11. When the most proximal preselector stop feature 73 is longitudinally aligned with the dose stop feature 63 the largest preselection indication 77, i.e. number 3 shows up in the preselection window 11.

Starting from the configuration of FIGS. 5 and 20 and when rotating the preselector 70 in a clockwise direction as seen from the proximal end of the injection device 1 the proximal most preselector stop feature 73 becomes aligned with the dose stop feature 63. Accordingly, a free path length for the longitudinal travel of the slider 60 between the initial position i and the activation position a will be enlarged. When finally arriving in the activation position a as illustrated in FIG. 21 the dose button 61 and hence the slider 60 protrudes even further from a proximal end of the housing 10 compared to the configuration of the preselector when another preselector stop feature 74 or 75 is aligned with the dose stop feature 63.

The housing 10 further comprises a dose indicating window 12 in which the momentary state or position of the slider 60 relative to the housing 10 is illustrated. In the dose indicating window 12 a dose size indicator 66 provided on an outside surface of the slider 60 shows up. When in the initial position as shown in FIG. 18 a dose size indicator 66 may show up in form of an arrow indicating to a user, that the slider 60 needs to be displaced towards the proximal direction 3. When reaching an activation position a as shown in any of the FIGS. 19 to 21 different or identical dose size indicators 66 will show up in the dose indicating window 12 thereby indicating to a user that the injection device 1 is ready for dispensing and for expelling of the dose of the medicament. Here, the dose size indicators 66 may show an arrow pointing in the distal direction 2.

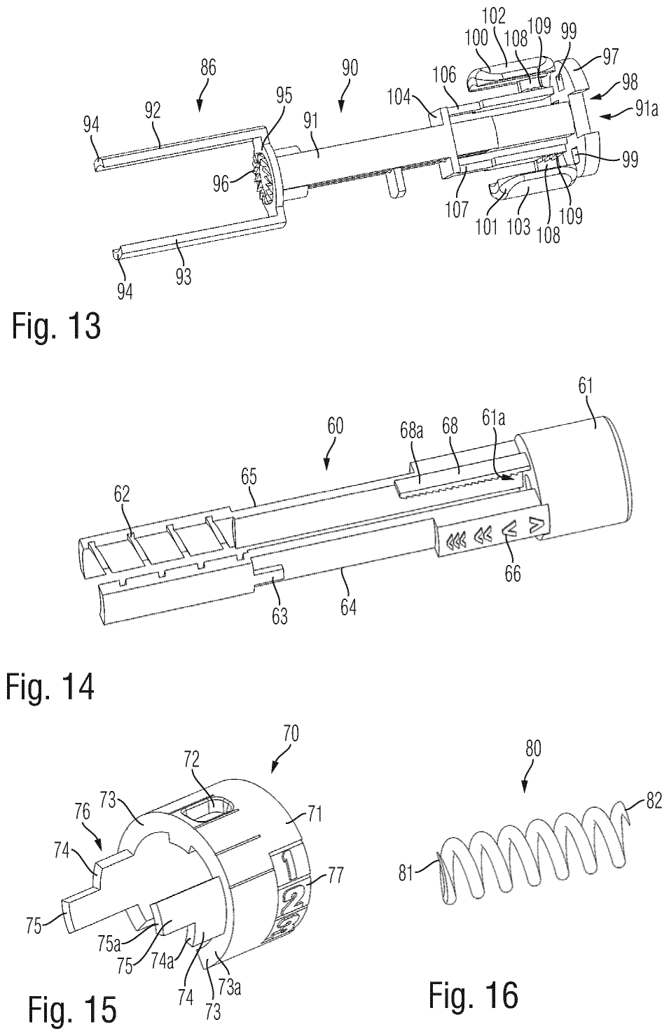

The injection device 1 further comprises a support 90 as shown in FIG. 13. The support 90 is fixed inside the housing 10. It serves as a mounting support or mounting platform for several other components of the drive mechanism 8. The support 90 may be also integrally formed with the housing 10. For the purpose of assembly of the injection device 1 it may be beneficial to provide the support 90 as a separate component to be assembled and fixed inside the housing 10. The support 90 further comprises a retainer 86 integrally formed with a support. First, the support 90 and the retainer 86 are positionally fixed inside the housing 10 of the injection device 1. The retainer 86 may be configured and designed as an integral component of the support 90. However, the retainer 86 is configured to mechanically engage with a driver 30 as will be explained below in greater detail. At least one of the support 90 and the retainer 86 may be also integrally formed with the housing 10.

The support 90 comprises a body 91 of elongated shape. Towards a proximal end the body 91 comprises a radially widened flange section 97 having two diametrically oppositely located recesses 98. The slider 60 comprises two elongated legs 64, 65, each of which being longitudinally guided in any one of the recesses 98. In this way the slider 60 is longitudinally displaceable relative to the housing 10 and relative to the support 90. The slider 60 is allowed to slide relative to the support 90 in longitudinal direction but is hindered to rotate relative to the support and/or relative to the housing 10.

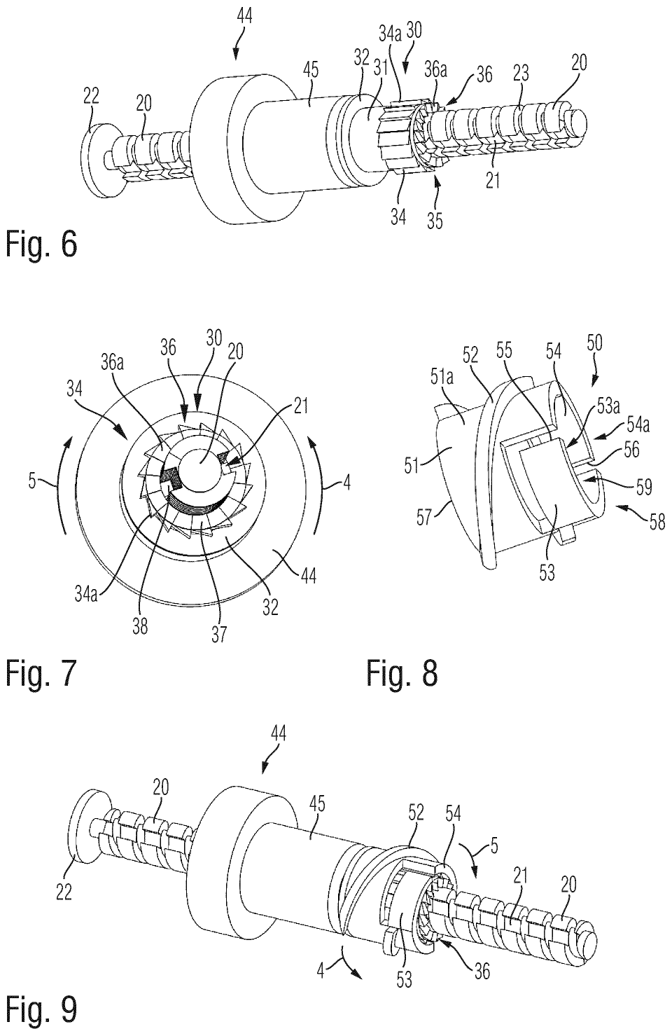

The support 90 comprises two geometrically opposed and longitudinally extending strut sections 92, 93 each of which having a distal face 94. In a final assembly configuration as for instance shown in FIG. 23 the strut sections 92, 93 are in axial abutment with a threaded insert 44 or with a radially inwardly extending flange section of the housing 10. The threaded insert 44 is separately illustrated in FIG. 6. It may be integrally formed with an inside facing portion of the sidewall 13 of the housing 10. The threaded insert 44 comprises a sleeve section 45 through which the piston rod 20 extends in longitudinal direction. The sleeve section 45 and hence the threaded insert 44 comprises an inner thread 43 that is in threaded engagement with an outer thread 23 of the piston rod 20.

The threaded insert 44 comprises a radially widening socket section 47 extending radially outwardly from the sleeve section 45. The socket section 47 is connected to the sidewall 13 of the housing 10. The socket section 47 forms and comprises a radially outwardly extending shoulder portion 48. As illustrated in FIG. 23 the distal faces 94 of the strut sections 92, 93 are in axial abutment with the shoulder portion 48. In this way the support 90 can be axially fixed inside the housing 10. The elongated legs 64, 65 of the slider 60 each comprise a distal face 67 that is configured to get in axial abutment with the shoulder portion 48 of the threaded insert 44 when arriving in the initial position i, e.g. at the end of a dose dispensing pocedure. In this way the distally directed displacement of the slider 60 can be blocked and limited thereby terminating a dose dispensing procedure.

The slider 60 further comprises a tubular or knob-like shaped dose button 61 having a distally facing support face 61a. The dose button 61a forms a proximal end of the slider 60. A distal end face of the dose button 61 may get in axial abutment with the flange section 97 of the support 90 as illustrated for instance in FIG. 23 in order to limit a distally directed displacement of the slider 60 and in order to define the initial position i of the slider 60.

In the initial position i as shown in FIG. 23 the support face 61a is in axial abutment with the flange section 97 of the support 90. Between the support 90 and the slider 60 there is provided the spring 80. As illustrated in FIG. 23 the support 90 comprises a central bore in which a distal end 81 of the spring 80 is located. An opposite end of the spring, hence a proximal end 82, is located inside a bore of the dose button 61. The distal end and/or the proximal end 81, 82 of the spring 80 are either fixed to the support 90 and to the slider 60 or they are in abutment with respective abutment faces of the support 90 and the slider 60.

The spring 80 comprises a helically wound compression spring. In the initial position of the slider 60 the spring 80 is pre-tensioned at least to a predefined degree such that upon release of the interlock 84 the slider 60 becomes subject to a proximally directed sliding motion relative to the support 90.

The interlock 84 is illustrated in greater detail in FIGS. 12, 17 and 23. It comprises a first engaging structure 68b, 69b connected to or integral with the slider 60 and a second engaging structure 109 connected to or integral with the at least one release member 100, 101. The slider 60 comprises two diametrically oppositely located and longitudinally extending interlock members 68, 69. The interlock members 68, 69 comprise longitudinally extending straight shaped arms or legs extending axially from a distal end of the dose button 61. The interlock members 68, 69 extend substantially parallel to the elongation of the legs 64, 65 of the slider 60. As seen in circumferential direction the two interlock members 68, 69 are located tangentially or circumferentially between the diametrically oppositely located legs 64, 65.

The interlock members 68, 69 each extend through another recess 99 or through opening provided in the flange section 97 at the proximal end of the support 90. As shown in FIG. 17 in greater detail the interlock members 68, 69 each comprise an elongated arm 68a, 69a. Each one of the interlock members 68, 69 comprises an engaging structure 68b, 69b. In the present example the engaging structures 68b, 69b comprise a serrated or toothed surface that is selectively engageable with a correspondingly shaped engaging section 109 of the release members 100, 101.

The release members 100, 101 may be integrally formed with the support 90. Alternative, they are provided as separate components. The release members 100, 101 and the respective release buttons 102, 104 are provided at a free end of resilient arms 106, 107 of the support 90, which arms 106, 107 are deflectable in radial direction. As illustrated in FIG. 13 the resilient arms 106, 107 are provided and arranged on a flange section 104 of the support 90 protruding radially outwardly from the body 91 of the support 90.

The resilient arms 106, 170 extend substantially parallel to the arms 68a, 69a of the interlock members 68, 69. That side of the resilient arm 106 facing towards the interlock member 68 is provided with an engaging structure 109 in form of a toothed section configured to releasably engage with the engaging structure 68b. That side of the resilient arm 107 facing towards the interlock member 69 is also provided with a correspondingly shaped engaging section, in form of a toothed section 109. The teeth of the engaging sections 68b, 69b, 109 comprise a saw tooth profile thus allowing a distally directed sliding displacement of the slider 60 relative to the release members 100, 101 and their respective resilient arms 106, 107.

The saw tooth profile of the engaging structures 68b, 69b, 109 is such, that the slider 60 and hence the interlock members 68, 69 thereof are hindered from a proximally directed sliding displacement as long as the release member 100, 101, the release buttons 102, 103 and the resilient arms 106, 107 are located in an initial and non-depressed configuration.

As illustrated further in FIG. 17 the interlock members 68, 69 and hence the elongated arms 68a, 69a extend in longitudinally direction between the release buttons 102, 103 and the respective resilient arms 106, 107. In other words the interlock members 68, 69 each extend through a gap between the toothed section 109 and the corresponding release button 102, 103. The release buttons 102, 103 are connected to the resilient arms 106, 107 by means of a radially extending connecting piece 108 as shown in FIG. 13. The radial extension of the connecting piece 108 is larger than a radial thickness of the interlock members 68, 69, respectively.

By simultaneously depressing both release members 100, 101 and hence both release buttons 102, 103 the respective resilient arms 106, 107 are displaced radially inwardly thereby disengaging the engaging sections 109 of the release members 100, 101 from the engaging sections 68b, 69b of the interlock members 68, 69, respectively. In this way the interlock 84 is released and the slider 60 is free to become displaced in proximal direction 3 under the action of the spring 80.

The support 90 further comprises a distally facing toothed section 96. The toothed section 96 may be provided in the region of or on a flange section 95 from which the two strut sections 92, 93 extend in distal direction 2. The toothed section 96 is of annular shape and faces in distal direction. The toothed section 96 comprises a saw-toothed profile.

The piston rod 20 comprises a pressure foot 22 that is rotationally supported on the distal end of the piston rod 20. In this way the piston rod 20 is allowed to rotate relative to the pressure foot 22 when the pressure foot 22 is in axial abutment with a proximal thrust receiving surface of the piston 7 of the cartridge 6. A detailed view of the piston rod is shown in FIG. 6. The piston rod 20 comprises an outer thread 23 that is threadedly engaged with the inner thread 43 of the threaded insert 44. Alternative, the piston rod 20 extends through a threaded bore of the housing 10. The piston rod 20 further comprises two elongated, straight shaped and axially extending grooves 21 intersecting the outer thread 23. As illustrated in FIG. 7 the oppositely located grooves 21 are in a splined engagement with radially inwardly extending protrusions 38 of a driver 30.

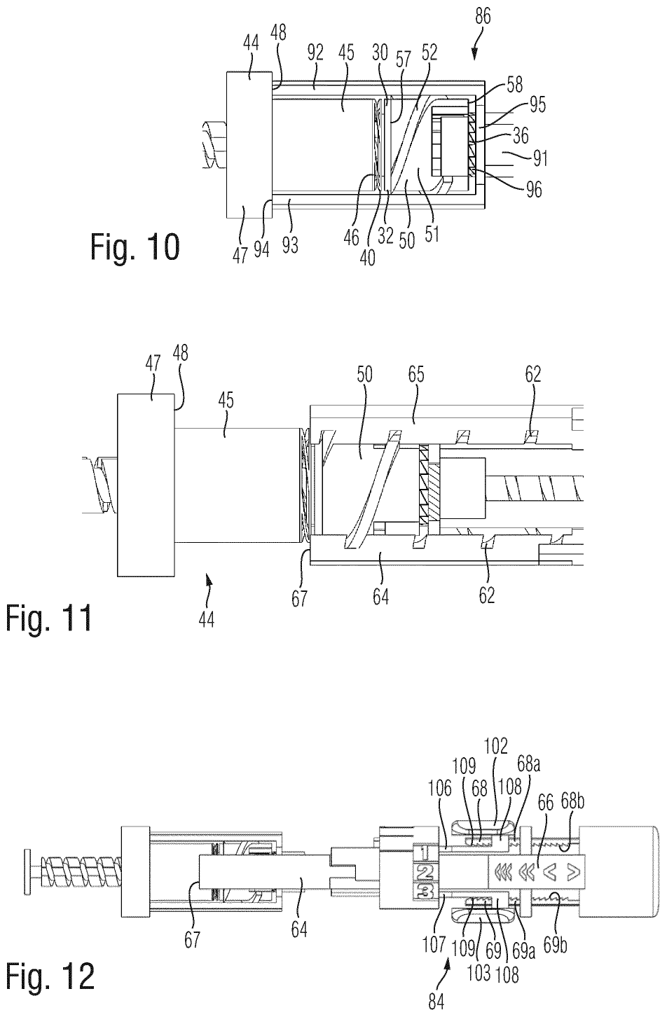

The driver 30 comprises a driver sleeve section 31 enclosing an axial portion of the piston rod 20. The driver 30 comprises a radially widened flange 32 near or at its distal end. The flange 32 is in axial abutment with a clutch spring 40. The clutch spring 40 as illustrated in FIG. 10 is axially sandwiched between a proximal face 46 of the threaded insert 44 and the distal end of the driver 30. The clutch spring 40 is configured or comprises a compression spring. One end of the clutch spring 40 is supported by the proximal face 46 of the threaded insert 44 and an opposite end of the clutch spring 40 is in abutment with the flange 32 of the driver 30. The distal end of the clutch spring 40 may be alternatively in abutment with a proximal face, with a rim or with a radially inwardly extending flange section of the housing 10.

As illustrated further in FIGS. 6, 7 and 9 the driver 30 comprises a first toothed section 36 and a second toothed section 34 at or near a proximal end of the driver 30. The first toothed section 36 is provided at an axial face 35, typically at an axial end face 35 of the driver. It is provided at a proximal axial end face. It is configured to engage with a correspondingly-shaped toothed section 96 of the retainer 86. The retainer 86 is separately illustrated in FIG. 13. The first toothed section 36 is of annular shape and comprises numerous teeth 36a that are arranged next to each other along the circumference of the driver sleeve section 31. Typically, the teeth 36a of the first toothed section 36 resemble or comprise a hirth toothing, wherein the tips of the teeth 36a protrude in axial direction and wherein the grooves between consecutive teeth 36a extend radially with regard to the tubular shape of the driver sleeve section 31.

The teeth 36a of the first toothed section 36 comprise a saw tooth profile. Hence, the teeth 36a of the first toothed section 36 each comprise a saw tooth 36a. The saw teeth 36a of the first toothed section 36 comprise a steep edge and a shallow or flat edge. As illustrated in FIGS. 6 and 9 the steep edges of the teeth 36a of the first toothed section 36 face towards a first sense of rotation or in a first direction 4. The shallow or flat edges of the saw teeth 36a of the first toothed section 36 faces towards a second sense of rotation or in a second direction 5 as illustrated in FIG. 9.

The retainer 86 comprises a correspondingly-shaped toothed section 96 as illustrated in FIGS. 10 and 13. The correspondingly-shaped toothed section 96 also comprises numerous saw teeth that are of substantially identical shape and size compared to the saw teeth 36a of the first toothed section 36. Since the driver 30 is biased by the clutch spring 40 in proximal direction 3 the first toothed section 36 provided at the proximal end face 35 of the driver 30 is kept in abutment and in engagement with the correspondingly-shaped toothed section 96 of the retainer 86. Due to the mutually corresponding saw toothed profiles of the first toothed section 36 and the correspondingly-shaped toothed section 96 a rotation of the driver 30 along the first direction 4 is permanently prevented. A rotation in the opposite, hence along the second direction 5 is allowed and supported.

When the driver 30 is rotated in the second direction 5 the shallow or flat-shaped edges of the first toothed section 36 and the correspondingly-shaped toothed section 96 are allowed to slide relative to each other. Such a rotational motion of the first toothed section 36 relative to the toothed section 96 of the retainer 86 may be accompanied by a slight axial displacement of the driver 30 in longitudinal direction (z).

As the shallow edges of the teeth 36a of the first toothed section 36 and the correspondingly-shaped toothed section 96 are subject to a relative sliding displacement in circumferential direction the axial slope of the saw toothed profiles of the teeth of the toothed sections 36, 96 leads to a distally directed sliding motion of the driver 30 until the crest or tips of the teeth of the mutually engaged toothed sections 36, 96 pass each other. As soon as the tips of the teeth of the mutually corresponding toothed sections 36, 96 have passed, the clutch spring 40 urges the driver 30 in proximal direction 3 so that the tips or crests of the toothed section 36 engage with the grooves of the correspondingly-shaped toothed section 96 and vice versa.

The rotation of the driver 30 along and in the second direction 5 may be thus accompanied by a back and forth movement of the driver 30 in longitudinal direction. The stepwise and ratchet-like rotational movement of the driver 30 relative to the retainer 86 and relative to the housing 10 may be further accompanied by an audible click sound thus providing an audible feedback to the user or healthcare giver that a dispensing or drug delivery operation is currently in process.

When the driver should become subject to a torque along the first direction 4 the steep edges of the teeth 36a of the first toothed section 36 are and remain in torque-proof engagement with correspondingly-shaped steep edges of the saw teeth of the correspondingly-shaped toothed section 96 of the retainer 86. In this way a rotation of the driver 30 along the first direction 4 is effectively prevented.

Since the driver 30 is in permanent splined engagement with the piston rod 20 through the protrusion 38 a back winding or retraction of the piston rod 20 along or in proximal direction 3 is effectively impeded and prevented. A rotation along the second direction 5 or along the second sense of rotation opposite to the first sense of rotation or direction 4 is supported and allowed by the engagement of the toothed sections 36, 96.

The injection device 1 further comprises a clutch 50 having a hollow interior 59. The clutch 50 is configured to receive at least a portion of the driver 30 inside the hollow interior 59. At least a portion of the driver sleeve section 31 and/or a portion of the driver 30 is arranged inside the hollow interior 59 of the clutch 50. In this way a nested or interleaved configuration of the driver 30 and the clutch 50 can be provided. This allows for a rather stable and robust construction of the drive mechanism 8 of the injection device 1.

Moreover, the at least partially nested or interleaved arrangement and configuration enables a rather compact and space saving design of the injection device 1. The partially interleaved or nested configuration is also beneficial in that the driver 30 and the clutch 50 provide mutual support with regard to a rotation relative to the housing 1. For instance, the driver 30 is mechanically supported by the piston rod 20 and the interleaved or nested arrangement between the driver 30 and the clutch 50 provides a rotational support for the clutch 50. Since the clutch 50 receives at least a portion of the driver sleeve section 31 the clutch 50 is rotationally supported by the driver 30. This is beneficial for a torque transmitting engagement between the clutch 50 and the driver 30 and may reduce mechanical tolerances and backlash between the various components of the injection device 1.

As shown in FIGS. 9 and 10 the clutch 50 and in particular a clutch sleeve section 51 thereof comprises a distal face 57 in axial abutment with the proximal side of the flange 32 of the driver 30. The clutch 50 further comprises an outer thread 52 that is in threaded engagement with an inner thread 62 provided on a section of the slider 60. The inner thread 62 is provided on and/or distributed on the two legs 64, 65 of the slider 60. In this way a longitudinal sliding displacement of the slider 60 is transferrable into a rotation of the clutch 50. The threaded engagement between the slider 60 and the clutch 50 is such that a proximally directed displacement of the slider 60 relative to the housing 10 or relative to the clutch 50 leads to a rotation of the clutch 50 along the first direction 4.

An oppositely directed sliding displacement of the slider 60 in distal direction 2 relative to the housing 10 and hence relative to the clutch 50 leads to a rotation of the clutch 50 along the second direction 5. The clutch 50 and the slider 60 are permanently threadedly engaged. Any axial sliding displacement of the slider 60 relative to the housing 10 and/or relative to the clutch 50 transfers into a respective rotation of the clutch 50 along the first direction or the second direction.

The clutch 50 is in unidirectional torque transmissive engagement with the driver 30. This is achieved by a second toothed section 34 provided on an outside surface of a sidewall 37 of the driver sleeve section 31. The second toothed section 34 also comprises numerous saw teeth 34a each of which protruding radially outwardly from the outside surface of the sidewall 37 as illustrated in FIG. 7. The saw teeth 34a each comprise a steep edge facing in or along the first direction 4. The saw teeth 34a also comprise a shallow or flat edge facing towards the second direction 5.

The second toothed section 34 may be arranged axially adjacent to the first toothed section. The steep and flat or shallow edges of the teeth 36a, 34a may be in radial alignment or may flush in radial direction. Hence, the first toothed section 36 and the second toothed section 34 comprise an equal number of consecutive teeth.

The clutch 50 comprises at least one engaging section 55, 56. Typically and as illustrated in FIG. 8 the clutch 50 comprises a first and a second engaging section 55, 56. The engaging sections 55, 56 are located at free ends 53a, 54a of a first and a second ratchet member 53, 54, respectively. Generally, the clutch 50 comprises at least one ratchet member 53, 54 that is resiliently deformable in radial direction. In the presently illustrated example the clutch 50 comprises two ratchet members, namely a first ratchet member 53 and a second ratchet member 54. The ratchet members 53, 54 are provided at a proximal end of the clutch 50 and hence at a proximal end of the clutch sleeve section 51.

A proximal face 58 of the clutch 50 is formed by or constituted by the first and the second ratchet members 53, 54. Each one of the at least first and second ratchet members 53, 54 comprises an arc-shaped geometry that is conformal to a sidewall 51a of the clutch sleeve section. Hence, the ratchet members 53, 54 are axially flush with the sidewall 51a of the clutch sleeve section 51. The ratchet members 53, 54 are integrally formed with the clutch 50 and hence with the clutch sleeve section 51. The clutch 50 may comprise or may consist of an injection molded plastic component.

The free ends 53a, 54a of the ratchet members 53, 54 are separated from the clutch sleeve section 51 by a longitudinal or L-shaped slit in the sidewall 51a of the clutch sleeve section 51. The engaging sections 55, 56 may comprise radially inwardly extending protrusions to engage with the steep edges of the saw teeth 34a of the second toothed section 34. However, it may be even sufficient that an end face of the ratchet members 53, 54 gets in engagement with the steep edges of the saw teeth 34a.

This may be attained when the outer diameter of the second toothed section 34 as measured at the tips of the saw teeth 34a is slightly larger than an inside diameter of the clutch sleeve section 51 in the region of the first and second ratchet members 53, 54. In this way the ratchet members 53, 54 are resiliently deformed radially outwardly when the second toothed section 34 is located in the free space between the at least two ratchet members 53, 54.

Alternatively it is conceivable, that the ratchet members 53, 54 are biased radially inwardly so that in an initial configuration the free ends of the ratchet members 53, 54 and hence the engaging sections 55, 56 thereof protrude radially inwardly from the inside surface of the sidewall 51a of the clutch sleeve section 51. As the clutch 50 receives the driver 30 the ratchet members 53, 54 will then be at least slightly biased radially outwardly when engaging with the second toothed section 34.

The present example shows resiliently deformable ratchet members 53, 54. However, the injection device 1 is by no way limited to resiliently deformable ratchet members. It is also conceivable, that the ratchet members 53, 54 are pivotally supported on the clutch 50. They may be pivotable radially outwardly against a restoring force that may be provided by a spring not further illustrated here. In this way, a similar ratchet effect could be attained.

As illustrated in FIG. 9 the engaging sections 55, 56 are in permanent engagement with the saw teeth 34a of the second toothed section 34 of the driver 30. The saw toothed profile of the second toothed section 34 is selected such, that the ratchet members 53, 54 of the clutch 50 slide along and relative to the second toothed section 34 as the clutch 50 is rotated along the first direction 4 during a dose setting procedure. Since the driver 30 is hindered to rotate along the first direction 4 through the engagement with the retainer 86 the driver 30 cannot follow the rotation of the clutch 50 that is induced by a proximally directed displacement of the slider 60.

The rotation of the clutch 50 along the first direction 4 is accompanied by an audible click sound that is generated as the ratchet members 53, 54 pass a tip of the teeth 34a of the second toothed section 34. A click sound is generated each time when an engaging section 55, 56 passes over a tip of a tooth 34a of the second toothed section 34 thereby providing an audible feedback to the user of the injection device 1 that a dose setting procedure is in progress. As the clutch 50 is rotated in the first direction 4 the driver 30 is and remains in torque-proof engagement with the retainer 86.

When the clutch 50 is subject to a rotation in the second direction 5 the engaging sections 55, 56 remain in abutment with the steep flange or steep edge of the teeth 34a of the second toothed section 34 thereby transferring a respective angular momentum or torque to the driver 30 along the second direction 5. Consequently, the driver 30 is rotated in the second direction 5 which rotation is equally transferred to a rotation of the piston rod 20.

Due to its threaded engagement with the threaded insert 44 the piston rod 20 advances in distal direction 2 so as to expel the set dose of the medicament from the cartridge 6.

The clutch 50 is axially or longitudinally sandwiched between the flange section 95 of the support 90 and the flange 32 of the driver 30. In this way the clutch 50 is axially constrained inside the housing 10. A proximal face 58 of the clutch 50 is in axial abutment with the support 90 or with the retainer 86. The distal face 57 of the clutch is in axial abutment with a proximal side of the flange 32 of the driver 30. When the slider 60 is depressed in distal direction 2 the clutch 50 may be subject to a distally directed displacement before it starts to rotate due to the threaded engagement with the slider 60.

The axially distally directed displacement of the clutch 50 at the beginning of a dose dispensing procedure is transferred to a respective axial displacement of the driver 30 since the distal face 57 of the clutch 50 is and remains in abutment with the proximal side of the flange 32. In this way the first toothed section 36 may disengage from the toothed section 96 of the retainer 86. Accordingly, the driver 30 may start to rotate along the second direction 5 while being out of contact with the retainer 86. Consequently, a dispensing force to be applied to the slider 60 in distal direction 2 can be decreased because there is no longer a friction between the first toothed section 36 and the correspondingly shaped toothed section 96 of the retainer 86 as long as the slider 60 is depressed, e.g. by a thumb of a user.

Operation of the injection device 1 is as follows. When handed out to a patient or consumer the injection device 1 may be ready for dispensing. The injection device may be preconfigured or manufactured in such a way that a priming procedure is not necessary. Alternatively, it is conceivable, that the injection device has to undergo a priming procedure or an air shot so as to make sure that the pressure foot 22 of the piston rod 20 is in direct abutment with the piston 7 of the cartridge 6.

A user has to depress the two release members 100, 101 simultaneously. In this way the two sections 109 thereof and the interlock members 68, 69 disengage and are operably released from each other. The slider 60 is then free to be displaced in proximal direction 3 under the action of the releasing spring 80. This proximally directed displacement of the slider 60 continues until the dose stop feature 63 of the slider 60 gets in axial abutment with one of the preselector stop features 73, 74, 75. Then and due to the proximally directed displacement of the slider 60 the dose button 61 thereof protrudes from a proximal end of the housing 10 as for instance illustrated in FIG. 20. The device is then ready for dispensing or for expelling of a dose of the medicament. In the preselection window 11 the preselected size of a dose is indicated. In the corresponding dose indicating window 12, e.g. two arrows show up thus indicating to the user that the dose button 61 can now be depressed in distal direction 2.

The proximal displacement of the slider 60 is accompanied by a rotation of the clutch 50 in the first sense of rotation 4 as illustrated in FIG. 9. The driver 30 is kept stationary and remains in non-rotational engagement with the support 90 by the toothed sections 36 and 96. This rotational interlock is further supported by the clutch spring 40 configured to urge the driver 30 in a unidirectional torque proof and non-rotative engagement with the support 90.

During a dose dispensing procedure in which the slider 60 is depressed in distal direction 2 against the action of the spring 80 the clutch 50 is subject to a rotation along the second sense of rotation 5. The ratchet members 53, 54 of the clutch 50 and their engaging sections 55, 56 are configured to transfer an angular momentum from the clutch 50 to the driver 30. Insofar the driver 30 also starts to rotate along the second sense of rotation 5. The radially inwardly extending protrusions 38 of the driver 30 are in splined engagement with respective longitudinal grooves 21 of the piston rod 20. A rotation of the driver 30 along the second sense of rotation 5 therefore transfers into a respective rotation of the piston rod 20. Due to the threaded engagement of the piston rod 20 with the housing 10 the piston rod 20 becomes subject to a respective distally directed advancing motion thereby expelling a respective amount of the medicament from the cartridge 6.

The longitudinal travel of the slider 60 relative to the housing 10 between the initial position i and a respective activation position a is determined by the positional state of the preselector 70.

The preselector 70 comprises at least one axially extending protrusion 76. As shown in FIG. 15 the preselector 70 may even comprise two diametrically oppositely located and symmetrically configured protrusions 76 each of which having numerous preselector stop features 75, 74. A bottom of the protrusion 76 and hence a rim of the sleeve section 71 of the preselector 70 may form or comprise another preselector stop feature 73. Each of the preselector stop features 73, 74, 75 comprises a well-defined stop face 73a, 74a, 75a. One of the stop faces 73a, 74a, 75a can be brought in axial alignment with the dose stop feature 63. The free space between the dose stop feature 63 and that particular stop face 73a, 74a, 75a that is in axial alignment with the dose stop feature 63 determines the axial distance that the slider 60 can be displaced between the initial position i and the at least one activation position a.

Modifying of a preselection of a dose requires a rotation of the preselector 70 with the longitudinal axis of the injection device as an axis of rotation. In this way another one of the preselector stop features 73, 74, 75 can be brought in longitudinal alignment with the dose stop feature 63. Since the axial positions of the preselector stop features 73, 74, 75 all differ, correspondingly modified longitudinal displacement paths of the slider 60 can be implemented.

### LIST OF REFERENCE NUMBERS

| | |
|---|---|
| 1 | injection device |
| 2 | distal direction |
| 3 | proximal direction |
| 4 | first direction |
| 5 | second direction |
| 6 | cartridge |
| 7 | piston |
| 8 | drive mechanism |
| 9 | dose setting mechanism |
| 10 | housing |
| 11 | preselection window |
| 12 | dose indicating window |
| 13 | sidewall |
| 14 | cartridge holder |
| 15 | needle assembly |
| 16 | inner needle cap |
| 17 | outer needle cap |
| 18 | protective cap |
| 19 | recess |
| 20 | piston rod |
| 21 | groove |
| 22 | pressure foot |
| 23 | thread |
| 25 | barrel |
| 26 | seal |
| 28 | socket |
| 30 | driver |
| 31 | driver sleeve section |
| 32 | flange |
| 33 | bore |
| 34 | toothed section |
| 34a | tooth |
| 35 | axial face |
| 36 | toothed section |
| 36a | tooth |
| 37 | sidewall |
| 38 | protrusion |
| 40 | clutch spring |
| 43 | inner thread |
| 44 | threaded insert |
| 45 | sleeve section |
| 46 | proximal face |
| 47 | socket section |
| 48 | shoulder portion |
| 50 | clutch |
| 51 | clutch sleeve section |
| 51a | sidewall |
| 52 | threaded section |
| 53 | ratchet member |
| 53a | free end |
| 54 | ratchet member |
| 54a | free end |
| 55 | engaging section |
| 56 | engaging section |
| 57 | distal face |
| 58 | proximal face |
| 60 | slider |
| 61 | dose button |
| 61a | support face |
| 62 | threaded section |
| 63 | dose stop feature |
| 63a | stop face |
| 64 | leg |
| 65 | leg |
| 66 | dose size indicator |
| 67 | distal face |
| 68 | interlock member |
| 68a | arm |
| 68b | engaging structure |
| 69 | interlock member |
| 69a | arm |
| 69b | engaging structure |
| 70 | preselector |
| 71 | sleeve section |
| 72 | recess |
| 73 | stop feature |
| 73a | stop face |
| 74 | stop feature |
| 74a | stop face |
| 75 | stop feature |
| 75a | stop face |
| 76 | protrusion |
| 77 | preselection indication |
| 78 | through opening |
| 80 | spring |
| 81 | distal end |
| 82 | proximal end |
| 84 | interlock |
| 86 | retainer |
| 90 | support |
| 91 | body |
| 92 | strut section |
| 93 | strut section |
| 94 | distal face |
| 95 | flange section |
| 96 | toothed section |
| 97 | flange section |
| 98 | recess |
| 99 | recess |
| 100 | release member |
| 101 | release member |
| 102 | release the button |
| 103 | release button |
| 104 | flange section |
| 106 | resilient arm |
| 107 | resilient arm |
| 108 | connecting piece |
| 109 | engaging structure |

1. An injection device for setting and injecting a dose of a medicament, the injection device comprising:
an elongated housing extending along a longitudinal axis;
a piston rod to operably engage with a piston of a cartridge filled with the medicament;
a clutch comprising a hollow interior and configured to be rotated in a first direction relative to the elongated housing during setting of the dose and to be rotated in a second direction relative to the elongated housing during delivery of the dose, wherein the second direction is opposite to the first direction;
a driver configured to follow a rotational movement of the clutch in the second direction during delivery of the dose, wherein the piston rod is operably engaged with the driver and is configured to be displaced along the longitudinal axis relative to the elongated housing when the driver rotates in the second direction; and
a retainer configured to prevent a rotational movement of the driver relative to the elongated housing in the first direction during setting of the dose,
wherein the driver comprises a driver sleeve section, and wherein at least a portion of the driver sleeve section is arranged inside the hollow interior of the clutch.

2. The injection device according to claim 1, wherein the clutch and the driver form a unidirectional torque transmission for the delivery of the dose, and wherein when the clutch is rotated in the second direction, the clutch is in torque-proof engagement with the driver (30).

3. The injection device according to claim 1, wherein the driver comprises a first toothed section at an axial face configured to engage with a correspondingly shaped toothed section of the retainer.

4. The injection device according to claim 3, wherein the first toothed section comprises a first plurality of saw teeth protruding in a longitudinal direction from the axial face of the driver.

5. The injection device according to claim 3, further comprising a clutch spring configured to urge the driver into abutment with the retainer.

6. The injection device according to claim 3, wherein the driver sleeve section comprises a sidewall with a second toothed section on an outside surface of the sidewall.

7. The injection device according to claim 6, wherein the second toothed section comprises a second plurality of saw teeth protruding radially from the outside surface of the sidewall.

8. The injection device according to 6, wherein the clutch comprises at least one engaging section configured for a unidirectional torque-proof engagement with a complementary or correspondingly shaped counter-engaging section of the driver.

9. The injection device according to claim 6, wherein the clutch comprises at least one engaging section configured for a unidirectional torque proof engagement with the second toothed section when the clutch is rotated in the second direction.

10. The injection device according to claim 9, wherein the clutch comprises at least one ratchet member resiliently deformable in a radial direction, and wherein the at least one engaging section is arranged at a free end of the at least one ratchet member.

11. The injection device according to claim 10, wherein the clutch comprises a clutch sleeve section enclosing at least a portion of the driver sleeve section of the driver.

12. The injection device according to claim 11, wherein the at least one ratchet member comprises an arc-shaped geometry conformal to a sidewall of the clutch sleeve section.

13. The injection device according to claim 10, wherein the clutch comprises at least a first ratchet member and a second ratchet member, and wherein the first ratchet member and the second ratchet member are arranged diametrically opposite to each other.

14. The injection device according to claim 1, wherein the driver is rotationally locked to the piston rod, and wherein the piston rod is threadedly engaged with the elongated housing.

15. The injection device according to claim 1, wherein the clutch comprises a threaded section that is in threaded engagement with a slider, wherein the slider is longitudinally slidably displaceable relative to the elongated housing, and wherein the slider is secured against rotational movement relative to the elongated housing.

16. The injection device according to claim 15, wherein the slider is biased in a proximal longitudinal direction by a spring.

17. The injection device according to claim 1, wherein the driver comprises a flange protruding radially outwardly from the driver sleeve section, and wherein the flange is in axial abutment with an axial face of the clutch.

18. The injection device according to claim 1, further comprising the cartridge.

19. The injection device according to claim 18, wherein the cartridge comprises a barrel filled with the medicament.

20. The injection device according to claim 19, wherein the barrel is sealed by a piston that is axially displaceable relative to the barrel by the piston rod.
